(19) United States
(12) Patent Application Publication
(10) Pub. No.: US 2014/0315850 A1
(43) Pub. Date: Oct. 23, 2014

(54) 2',3'-Dideoxy-2'-alpha-Fluoro-2'-beta-C-Methylnucleosides and Prodrugs Thereof

(71) Applicants: Qiang Huang, Suzhou (CN); Suping Zhou, Philadelphia, PA (US); Junbiao Chang, Henan (CN)

(72) Inventors: Qiang Huang, Suzhou (CN); Suping Zhou, Philadelphia, PA (US); Junbiao Chang, Henan (CN)

(73) Assignee: Nanjing Molecular Research, Inc., Nanjing, Jiangsu (CN)

(21) Appl. No.: 14/233,641

(22) PCT Filed: Jul. 19, 2012
(86) PCT No.: PCT/US12/47338

§ 371 Date: Jan. 17, 2014

Related U.S. Application Data
(60) Provisional application No. 61/509,478, filed on Jul. 19, 2011.

(51) Int. Cl. C07H 19/20; A61K 31/708; A61K 45/06; A61K 31/7076

(52) U.S. Cl. CPC C07H 19/20; A61K 31/7076; A61K 31/708; A61K 45/06
USPC 514/48; 536/26.7; 536/26.71; 536/4.1

(57) ABSTRACT

The present invention is made to fulfill the foregoing need. Since most of antiHN nucleosides are 2′,3′-dideoxynucleosides that have been proved to be excellent substrates of kinases for the phosphorylations. 2′,3′-Dideoxy-2,-a-fluoro-2′-{3-C-methyl-nucleosides can be considered as one unique class of 2′,3′-dideoxynucleosides to be good substrate of kinases because fluorine mimics hydrogen. It also can be considered as ribo-nucleosides to incorporate into RNA of HCV because 2′-fluorine-a mimics 2′-a-OH group.

### CROSS-REFERENCE TO RELATED APPLICATIONS

This application claims priority under 35 U.S.C. §119(e) to U.S. Provisional Patent Application Ser. No. 61/509,478, filed on Jul. 19, 2011, which is hereby incorporated by reference in its entirety.

### FIELD OF THE INVENTION

The present invention relates to 2′,3′-dideoxy-2′-α-fluoro-2′-β-C-methylnucleosides and their prodrugs, and therapeutic use thereof for treatment of hepatitis C virus (HCV) infections. The present invention also relates to processes and intermediates for the preparation of the nucleosides disclosed herein.

### BACKGROUND OF THE INVENTION

Hepatitis C virus (HCV) is a positive-stranded RNA virus. It is estimated that there are about 170 million of people infected with HCV in the world. Until May 2011, the treatment that has been approved by the US FDA for chronic HCV is pegylated interferon-α in combination with ribavirin as standard of care (SOC). Unfortunately, this treatment has limited efficacy with response rates of only 40-50% for the genotype-1 infected population, which is the most prevalent genotype in the US and China. In May 2011, FDA approved Incivek (Vertex) and Victelis (Merck) for the treatment of HCV infection in combination with SOC. These combinations may improve the response rates to high 60% to high 70%. However, their clinical usefulness is still limited by their serious side effects including depression, anaemia, and rashes.

A number of potential molecular targets for drug development of direct acting anti-HCV agents have been identified including the NS2-NS3 autoprotease, the N3 protease, the N3 helicase and the NS5B polymerase. The NS5B RNA-dependent RNA polymerase (RdRp) is essential for replication of the single-stranded, positive sense RNA genome, and this enzyme has attracted significant interest among medicinal chemists. Nucleosides active against HCV are the inhibitors of NS5B RdRp. Nucleosides have to be converted to their corresponding triphosphates that incorporate into viral RNA at 3′-terminal so as to stop viral RNA elongation as chain terminator.

Some nucleosides are weakly active because they cannot be efficiently phosphorylated by kinases or are not substrates of kinases at all, as some inactive nucleosides, when converted chemically to their triphosphates, become potently active against certain viruses in vitro. Nucleoside phosphates (nucleotides) per se cannot be used as drugs very often because they are de-phosphorylated by membrane nucleotides and other hydrolases before entering the cells or are too polar to enter the cells. To improve the biological activity of nucleosides, their phosphate prodrugs have been studied because they can potentially bypass the rate-limiting first step phosphorylation. Recently, the phosphoramidate prodrug approach has been proved to be an effective method for converting biologically inactive nucleosides into active nucleoside monophosphate bypassing the rate-limiting first step of phosphorylation (J. Med. Chem., 2007, 50(22), 5463-5470). Nucleoside phosphoramidate has been reported to efficiently deliver nucleoside 5′-monophosphate into the liver (WO 2008/121634; WO 2008/082601 and WO 2008/082602). In recent years, there have been a number of patent applications disclosing utilization of the phosphoramidates as prodrugs of nucleosides to deliver nucleoside monophosphates to tissues, in particular to the liver (U.S. Pat. No. 6,455,513, WO 2009/052050, WO 2008/121634, WO 2008/0833101, WO 2008/062206, WO 2007/002931, WO 2008/085508, WO 2007/095269, WO 2006/012078, WO 2006/100439). The nucleoside monophosphates can be further phosphorylated to di-, and then biologically active triphosphate. Therefore, phosphate prodrugs of the synthesized nucleosides were investigated together.

Two classes of nucleosides or nucleotides, 2′- and 4′-modified nucleosides or nucleotides have been identified as anti-HCV agents. Several of them have been advanced into various stages of clinical trials. Phase II clinical trials for R1626 and NM-283 had been terminated due to their serious side effects including GI toxicity and anaemia, respectively. Another five candidates, namely R7128 (U.S. Pat. No. 7,429,572), PSI-7977 (U.S. Pat. No. 7,964,580), PSI-938 (WO/2009/152095), IDX-184 (WO/2008/082601) and INX-189 (WO/2010/081082) demonstrated promising anti-HCV efficacy and a higher barrier to viral resistance in vitro and in clinical trials.

These nucleoside and nucleotides act as non-obligate chain terminators of HCV RdRp because they all have 3′-OH. Obligate chain-terminators that inhibit HCV replication have yet not been developed so far, perhaps because the presence of 3′-hydroxy group is a crucial structural determinant for the intracellular phosphorylation of ribo-nucleosides.

Most of potent antiviral nucleosides, such as FTC, 2′,3′-dideoxynucleosides, 2′,3′-dideoxy-2′,3′-didehydronucleosides, exert their effect for the treatment of HBV and HIV because they cannot support the elongation of the newly synthesized viral polynucleotide due to their lack of 3′-OH. 2′-C-Methylnucleosides and 4′-azidonucleosides demonstrated their anti-HCV activity probably due to their stereo-hindrance at the 2′- or 4′-position which reduces the possibility of chain elongation of newly synthesized viral polynucleotide at the 3′-OH. If nucleosides without 3′-OH showed potent anti-HCV activity, they would act as obligate chain terminators of viral RNA. However, ribo-nucleosides without 3′-OH may not be good substrates of kinases for the phosphorylation (J. Med. Chem. 2004, 47, 5041). Some groups investigated 3′-deoxynucleosides and their phosphate prodrugs as potential anti-HCV agents (Antivir. Chem. Chemother. 2002, 13, 363; Antivir. Res. 2003, 58. 243; Collet. Czech. Chem. Comm. 2006, 71, 991). The loss of anti-HCV activity of 3′-deoxy-2′-C-methylcytidine may be because its lack of 3′-OH resulted in inefficiency of phosphorylation of this ribo-nucleosides (Antiviral Res. 2003, 58, 243; Antimicrob. Agents Chemother. 2005, 49, 2050; Antimicrob. Agents Chemother. 2007, 51, 2920).

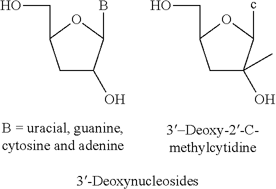

2′-Deoxy-2′-α-fluoro-2′-β-C-methylnucleosides demonstrated potent anti-HCV activity in vitro, in vivo and in clinic. However, their 3′-deoxynucleoside analogs have not been reported as potent anti-HCV agents in literature, and the related patent applications (Pharmasset: U.S. Pat. No. 7,429,572 and WO/2010/075549; Idenix: U.S. Pat. No. 7,547,704 and U.S. Pat. No. 7,608,600, Merck: U.S. Pat. No. 7,105,499 and U.S. Pat. No. 6,777,395) only claimed nucleosides with 3′-OH as anti-HCV agents probably due to the lack of successful examples of 3′-deoxynucleosides. Therefore, there is a need to develop 3′-deoxynucleoside analogs as efficient anti-HCV agents.

### SUMMARY OF THE INVENTION

The present invention is made to fulfill the foregoing need. Since most of anti-HW nucleosides are 2′,3′-dideoxynucleosides that have been proved to be excellent substrates of kinases for the phosphorylations. 2′,3′-Dideoxy-2′-α-fluoro-2′-β-C-methyl-nucleosides can be considered as one unique class of 2′,3′-dideoxynucleosides to be good substrate of kinases because fluorine mimics hydrogen. It also can be considered as ribo-nucleosides to incorporate into RNA of HCV because 2′-fluorine-α mimics 2′-α-OH group.

We synthesized 2′,3′-dideoxy-2′-α-fluoro-2′-β-C-methylnucleosides with modified guanine base and their phosphoramidate prodrugs, and evaluated their anti-HCV activity in Replicon. It was discovered that phosphoramidates of 2′,3′-dideoxy-2′-α-fluoro-2′-β-C-methylnucleoside analogs of guanosine demonstrated potent anti-HCV activity without significant cytotoxicity in vitro. Thus, in one aspect the present invention provides 2′,3′-dideoxy-2′-α-fluoro-2′-β-C-methylnucleosides and their phosphate prodrugs, and composition thereof for the treatment of HCV infection in human. In another aspect the present invention provides processes and intermediates for the preparation of 2′,3′-dideoxy-2′-α-fluoro-2′-β-C-methylnucleosides.

The present invention relates to 2′,3′-dideoxy-2′-α-fluoro-2′-β-C-methylnucleosides and their phosphate prodrugs, and the composition thereof for the treatment of HCV infection in humans. The present invention also relates to process and intermediates for the preparation of 2′,3′-dideoxy-2′-α-fluoro-2′-β-C-methylnucleosides. In particular, the present invention provides a compound of formula I:

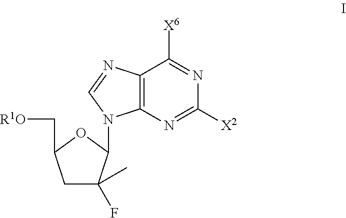

or its pharmaceutically acceptable prodrug, salt, solvate, a stereoisomic, tautomeric or polymorphic form, a metabolite thereof,

wherein:

R¹ is selected from H, monophosphate, diphosphate, triphosphate or their stable phosphate prodrugs, acyl (R²CO), R²OCO, and R²NHCO, R^{a}R^{b}NCO wherein: R^{a} and R^{b} are independently selected from alkyl, alkenyl, alkynyl, aryl, benzyl, cyclic alkyl, heterocyclyl, and heteroaromatic groups; R^{a}R^{b}N can be amino acid residue; R^{a} and R^{b}, together with the nitrogen atom, can form a 4- to 7-membered ring;

X² is selected from H, NH₂, and halogen (I, Br, Cl, F);

X⁶ is selected from H, OH, OMe, OEt, SMe, alkyloxy, aryloxy, cyclic alkyloxy, alkylthio, arylthio, cyclic alkylthio, thienyl, furyl, alkylamino, dialkylamino, arylamino, diarylamino, aryl alkylamino, cyclic alkylamino, and cyclopropylamino, wherein the dialkyl portion of the dialkylamino group, together with the nitrogen atom of the amino group, can optionally form a ring, such as azetidine;

amino and/or hydroxyl groups of above selected compounds are optionally protected.

In another aspect, the present invention provides compound and composition for the treatment of HCV infection in humans.

In another aspect, the present invention provides obligate chain terminators of NS5B polymerase of hepatitis C virus (HCV).

In another aspect, the present invention provides a method for the treatment of HCV infection by administering an effective amount of compound disclosed herein to patient alone or in combination with other antiviral agents.

In another aspect, the present invention provides process and intermediates for the preparation of compound disclosed herein.

### DETAILED DESCRIPTION OF THE INVENTION

The present invention relates to 2′,3′-dideoxy-2′-α-fluoro-2′-β-C-methylnucleosides and their phosphate prodrugs, and the composition thereof for the treatment of HCV infection in humans. The present invention also relates to process and intermediates for the preparation of 2′,3′-dideoxy-2′-α-fluoro-2′-β-C-methylnucleosides.

In one embodiment, the present invention provides a compound of formula I:

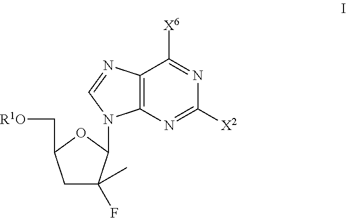

or its pharmaceutically acceptable prodrug, salt, solvate, a stereoisomic, tautomeric or polymorphic form, a metabolite thereof,

wherein: R¹ is selected from H, monophosphate, diphosphate, triphosphate or their stable phosphate prodrugs, acyl (R²CO), R²OCO, and R²NHCO, R^{a}R^{b}NCO wherein: R^{a} and R^{b} are independently selected from alkyl, alkenyl, alkynyl, aryl, benzyl, cyclic alkyl, heterocyclyl, and heteroaromatic groups; R^{a}R^{b}N can be amino acid residue; R^{a} and R^{b}, together with the nitrogen atom, can form a 4- to 7-membered ring;

X² is selected from H, NH₂, or halogen (I, Br, Cl, F);

X⁶ is selected from H, OH, OMe, OEt, SMe, alkyloxy, aryloxy, cyclic alkyloxy, alkylthio, arylthio, cyclic alkylthio, thienyl, furyl, alkylamino, dialkylamino, arylamino, diarylamino, aryl alkylamino, cyclic alkylamino, cyclopropylamino, dialkyl of dialkylamino can form a ring, such as azetidine; and

amino and/or hydroxyl groups of above selected compounds are optionally protected.

In the second embodiment, a stable phosphate prodrug of compound of formula I is selected from compounds of formulae IIa-c:

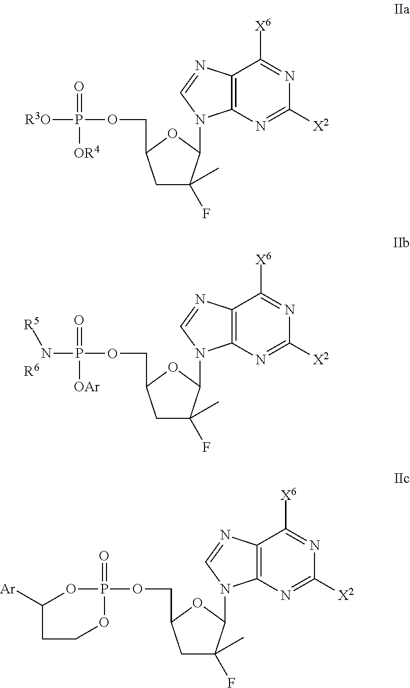

wherein:

X² and X⁶ are defined as above;

R³ and R⁴ are independently selected from alkyl, cyclic alkyl, aryl and benzyl or

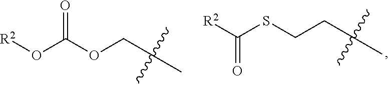

wherein: R² is defined as above;

Ar is aryl selected from unsubstituted or substituted heteroaromatic and aromatic groups, including but not limited to phenyl and naphthyl groups;

R⁵ and R⁶ are independently selected from alkyl, alkenyl, alkynyl, aryl, benzyl, cyclic alkyl, heterocyclyl, and heteroaromatic groups; R⁵ and R⁶, together with the nitrogen atom, can optionally form a 4- to 7-membered ring;

R⁵R⁶N can also be amino acid residue and aminoalcohol derivative of formulae:

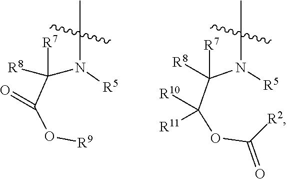

wherein:

R² is defined as above;

R⁷, R⁸, R⁹, R¹⁰ and R¹¹ are independently selected from alkyl, alkenyl, alkynyl, aryl, cyclic alkyl, heterocyclyl, and heteroaromatic groups; and

R⁷, R⁸ and R¹⁰, R¹¹ can independently, together with the carbon atom they attach to, form a 3- to 7-membered ring.

In the third embodiment, a stable phosphate prodrug of compound of formula III is one or a mixture of diastereomers of formula III:

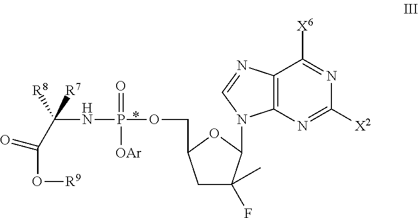

wherein: Ar, R⁷, R⁸, R⁹, X² and X⁶ are defined as above; and

symbol * represents for a chiral center.

In the forth embodiment, a stable phosphate prodrug of compound of formula IIb or III is selected from diastereomeric compounds of formulae IVa and IVb:

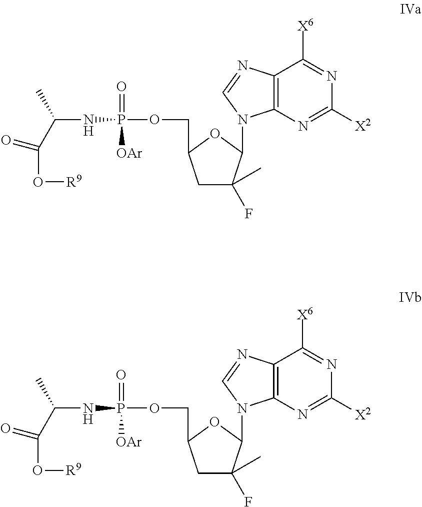

wherein: Ar, R⁹, X² and X⁶ are defined as above.

In the fifth embodiment, a stable phosphate prodrug of compound of formula IVa is selected from a compound of formulae:

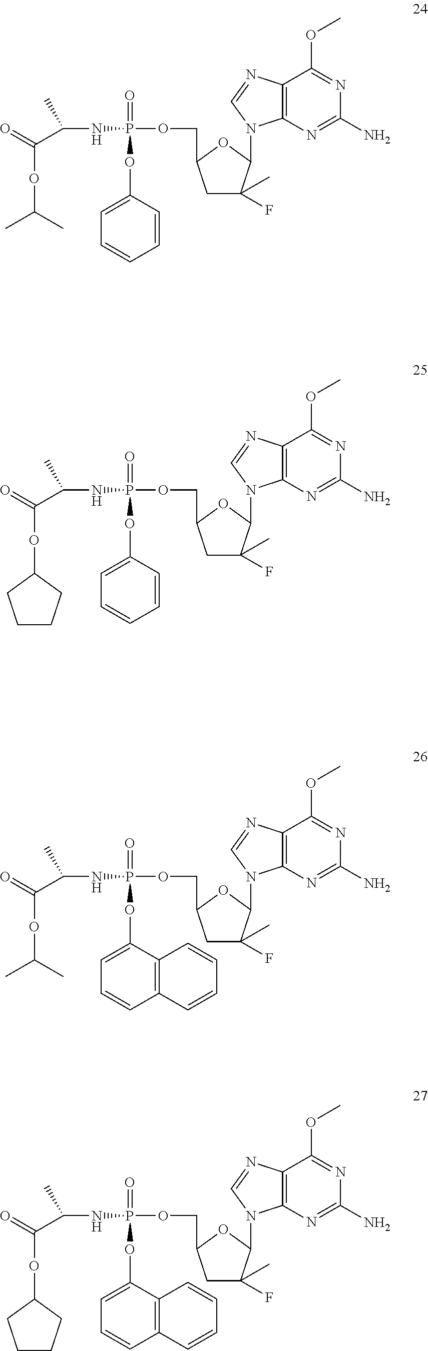 [SMILES: COc1nc(N)nc2c1ncn2C1OC(CO[P@@](=O)(N[C@@H](C)C(=O)OC(C)C)Oc2ccccc2)CC1(C)F ; COc1nc(N)nc2c1ncn2C1OC(CO[P@@](=O)(N[C@@H](C)C(=O)OC2CCCC2)Oc2ccccc2)CC1(C)F ; COc1nc(N)nc2c1ncn2C1OC(CO[P@@](=O)(N[C@@H](C)C(=O)OC(C)C)Oc2cccc3ccccc23)CC1(C)F ; COc1nc(N)nc2c1ncn2C1OC(CO[P@@](=O)(N[C@@H](C)C(=O)OC2CCCC2)Oc2cccc3ccccc23)CC1(C)F]

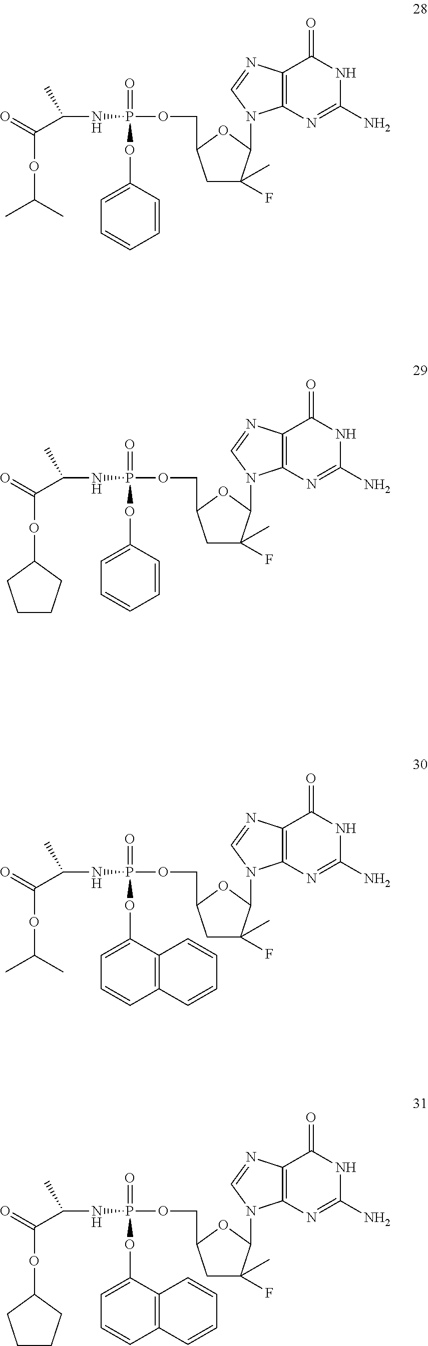 [SMILES: CC(C)OC(=O)[C@H](C)N[P@](=O)(OCC1CC(C)(F)C(n2cnc3c(=O)[nH]c(N)nc32)O1)Oc1ccccc1 ; C[C@H](N[P@](=O)(OCC1CC(C)(F)C(n2cnc3c(=O)[nH]c(N)nc32)O1)Oc1ccccc1)C(=O)OC1CCCC1 ; CC(C)OC(=O)[C@H](C)N[P@](=O)(OCC1CC(C)(F)C(n2cnc3c(=O)[nH]c(N)nc32)O1)Oc1cccc2ccccc12 ; C[C@H](N[P@](=O)(OCC1CC(C)(F)C(n2cnc3c(=O)[nH]c(N)nc32)O1)Oc1cccc2ccccc12)C(=O)OC1CCCC1]

### Therapeutic Use

In the sixth embodiment, 2′,3′-Dideoxy-2′-α-fluoro-2′-β-C-methylnucleosides and their phosphate prodrugs, and compositions thereof are provided. Therapeutic use of the nucleosides and their phosphate prodrugs, as well as compositions thereof is also provided for the treatment of HCV infection. Compounds disclosed herein and compositions thereof can be administered either alone or in combination with other therapeutically effective agents for the treatment of HCV infection.

### DEFINITIONS

When referring to the compounds provided herein, the following terms have the following meanings unless indicated otherwise.

The term “acyl” or “O-linked ester” includes a group of the formula of alkyl-CO or aryl-CO or cyclic alkyl-CO.

The term “alkyl”, as used herein, includes a saturated straight, branched, or cyclic, hydrocarbon of typically C₁ to C₂₀, and specifically includes methyl, CF₃, CCl₃, CFCl₂, CF₂Cl, ethyl, CH₂CF₃, CF₂CF₃, propyl, isopropyl, cyclopropyl, and the like. Non-limiting examples of moieties with which the alkyl group can be substituted are selected from the group consisting of halogen (fluoro, chloro, bromo or iodo), hydroxyl, amino, alkylamino, arylamino, alkoxy, aryloxy, nitro, cyano and the like.

“Alkenyl” includes monovalent olefinic unsaturated hydrocarbon groups, in certain embodiment, having up to 11 carbon atoms, which can be straight-chained or branched and having at least 1 or from 1 to 2 sites of olefinic unsaturation. Exemplary alkenyl groups include ethenyl (—CH═CH₂), n-propenyl (—CH₂CH═CH₂), isopropenyl (—C(CH₃)═CH₂), vinyl and substituted vinyl, and the like.

“Alkynyl” includes acetylenic unsaturated hydrocarbon groups, in certain embodiments, having up to about 11 carbon atoms which can be straight-chained or branched and having at least 1 or from 1 to 2 sites of alkynyl unsaturation. Non-limiting examples of alkynyl groups include acetylenic, ethynyl, propargyl, and the like.

The term “aryl”, as used herein, includes phenyl, biphenyl, or naphthyl, and preferably phenyl. The term includes both substituted and unsubstituted moieties. The aryl group can be substituted with any described moiety, including, but not limited to, one or more moieties selected from the group consisting of halogen (fluoro, chloro, bromo or iodo), alkyl, hydroxyl, amino, alkylamino, arylamino, alkoxy, aryloxy, nitro, cyano, sulfono, sulfato, phosphono, phosphato, or phosphonoxy, either unprotected, or protected as necessary.

“Cyclic alkyl” or cycloalkyl includes 3-7 membered rings of hydrocarbon, such as cyclopropyl.

“Heterocycles” includes 3-7 membered rings of carbon compounds with 1-3 heteroatoms, such as O, S, N in the ring.

“Heteroaromatic group” includes aromatic ring containing one to three heteroatoms, such as O, S, N, for example, pyridinyl, pyrimidinyl.

“Alkoxy or alkyloxy” includes the group —OR where R is alkyl. Particular alkoxy groups include n-pentoxy, n-hexoxy, 1,2-dimethylbutoxy, and the like.

“Amino” includes the radical —NH₂.

The term “alkylamino” or “arylamino” includes an amino group that has one or two alkyl or aryl substituents, respectively.

“Halogen” or “halo” includes chloro (Cl), bromo (Br), fluoro (F) or iodo (I).

“Monoalkylamino” includes the group alkyl-NHR′—, wherein R′ is selected from alkyl or aryl.

“Alkylthio” includes the group —SR where R is alkyl or aryl.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis. Protection and deprotection of functional groups in the processes below may be carried out by procedures generally known in the art (see, for example, T. W. Greene & P. G. M. Wuts, “Protecting Groups in Organic Synthesis”, 3^{rd} Edition, Wiley, 1999), which is hereby incorporated by reference. Examples of “protecting group” of oxygen or nitrogen include, but are not limited to, acyl (e.g., acetyl, formyl, benzoyl, etc.), carbonate (e.g., ROC(O)—, where R can be substituted or unsubstituted alkyl, alkenyl, aryl, benzyl, or the like), carbamate (e.g., R^{a}R^{b}N—C(O)—, wherein R^{a} and R^{b} are each independently hydrogen, alkyl, aryl, or the like). The oxygen and nitrogen protecting groups may also include unsubstituted or substituted benzyl groups, allyl, t-butyl groups, or silyl groups, which can be removed readily by methods well known in the art. In particular, suitable nitrogen protecting group is exemplified by benzyl- [Bn], tert-butoxycarbonyl-[BOC], tert-butyldimethylsilyl- [TBDMS], or the like.

The term “leaving group”, as used herein, refers to a group that can be replaced by another through a reaction such as displacement. Suitable leaving groups include, but are not limited to, halogen (Cl, Br, I) and sulfonates (—OS(O)₂-aryl (e.g., —OS(O)₂Ph or —OS(O)₂C₆H₄CH₃-p), or —OS(O)₂-alkyl (e.g., —OS(O)₂CH₃ or —OS(O)₂CF₃)), or the like.

“Pharmaceutically acceptable salt” includes any salt of a compound provided herein which retains its biological properties and which is not toxic or otherwise undesirable for pharmaceutical use.

The term “prodrug” as used herein refers to any compound that generates a biologically active compound when administered to a biological system as the result of spontaneous chemical reaction(s), enzyme catalyzed reaction(s), and/or metabolic process(es) or a combination of each. Standard prodrugs are formed using groups attached to functionality, e.g. —OH, —NH₂, —P(O)(NH)(OH), —P(O)(OH)₂, associated with the drug, that cleave in vivo. The prodrugs described in the present invention are exemplary, but not limited to, and one skilled in the art could prepare other known varieties of prodrugs.

The term “nucleoside” refers to a purine or pyrimidine base, or analogs thereof, connected to a sugar, including heterocyclic and carbocyclic analogues thereof.

The term “phosphate” refers to —O—PO₃²⁻. The term “phosphoramidate” refers to —N(R)—PO₃²⁻, wherein R is a hydrogen or a carbon-based substituent.

The term “biologically active drug or agent” refers to the chemical entity that produces the biological effect. In this invention, biolgically active agents refer to nucleoside, nucleoside mono-phosphates, nucleoside diphosphates, nucleoside triphosphates.

The term “alkaryl” or “alkylaryl” includes an aryl group with an alkyl substituent. The term aralkyl or arylalkyl includes an alkyl group with an aryl substituent.

The term “amino acid” includes naturally occurring and synthetic α-, β-, γ- or δ-amino acids, and includes but is not limited to, amino acids found in proteins, i.e.

glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In a preferred embodiment, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In one embodiment, a therapeutic agent is an agent which is known to be useful for, or has been or is currently being used for the treatment or prevention of a disorder or one or more symptoms thereof.

“Therapeutically effective amount” includes an amount of a compound or composition that, when administered to a subject for treating a disease, is sufficient to effect such treatment for the disease.

“Treating” or “treatment” of any disease or disorder refers, in one embodiment, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

### Preparation of Compounds

The compounds provided herein can be prepared, isolated or obtained by any method apparent to those of skill in the art. Exemplary methods of preparation are described in detail in the Examples Section below. Exemplary preparation of 2′,3′-dideoxy-2′-α-fluoro-2′-β-C-methylnucleosides is illustrated in Schemes 1 and 2. Compound 1 was prepared from 1,2; 5,6-diisopropylidinyl-D-manitol. Reaction of compound 1 with Wittig reagent gave compound 2 which was converted to compound 3 by hydrogenation in the presence of Pd/C. Treatment of compound 3 with HCl in aq EtOH followed by TBDPSCl protection of primary hydroxyl group produced compound 4. Treatment of a solution of compound 4 and (PhSO₂)₂ NF in THF with LiHMDS afforded compound 5 with the desired chirarity, exclusively. It was reported that fluorine attacked enolate intermediate from opposite side of silyloxymethyl group due to its bulkiness to generate single α-fluorinated precursor (J. Org. Chem. 1998, 63, 2161). Desilylation of compound 5 with TBAF followed by benzoylation with BzCl provided compound 7. Key intermediate lactol 8 was obtained by reduction of compound 7 by reducing agent, such as Li(t-BuO)₃AlH. The lactol 8 was converted to α-bromosugar 9 by the treatment of compound 8 with Ph₃P/CBr₄ (Scheme 2). The bromosugar then reacted with 6-chloro-2-aminopurine in the presence of base, such as t-BuOK, to give β-nucleoside 10 selectively. The final nucleoside 11 was obtained by treatment of compound 10 with MeONa in MeOH.

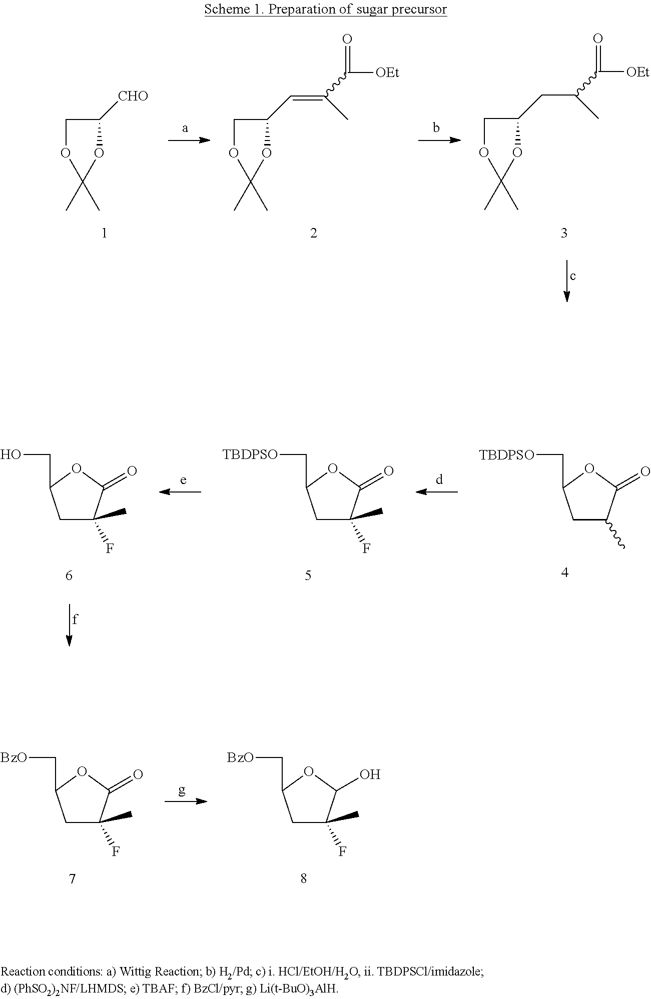

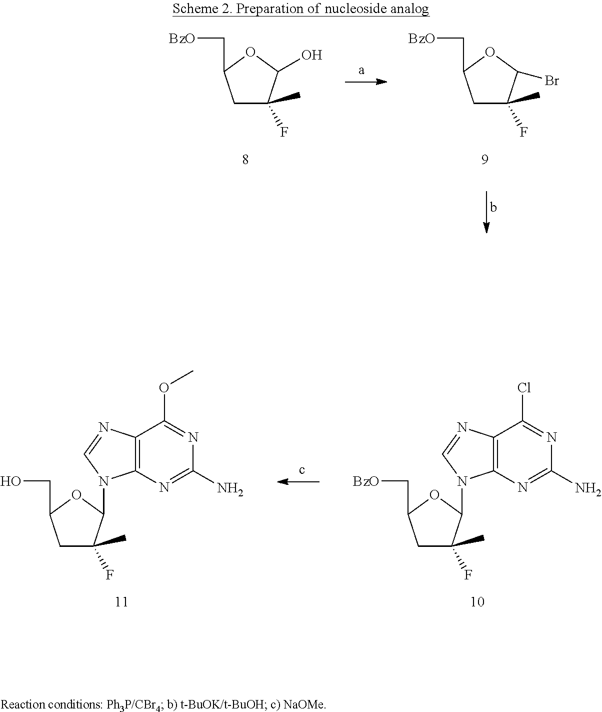

Preparation of compound 11 was also accomplished from nucleoside precursor with 3′-OH by 3′-deoxygenation (Scheme 3). Compound 12 was prepared according to method disclosed in patent application (WO/2010/075550). Treatment of 12 with

NaOMe in MeOH provided nucleoside 13. Selective protection of compound 13 with DMTrCl in pyridine gave compound 14. Treatment of 14 with PhOCSCl in the presence of Et₃N/DMAP in ACN followed by deoxygenation with Bu₃SnH/AIBN provided 3′-deoxynucleoside 11 after deprotection with TFA.

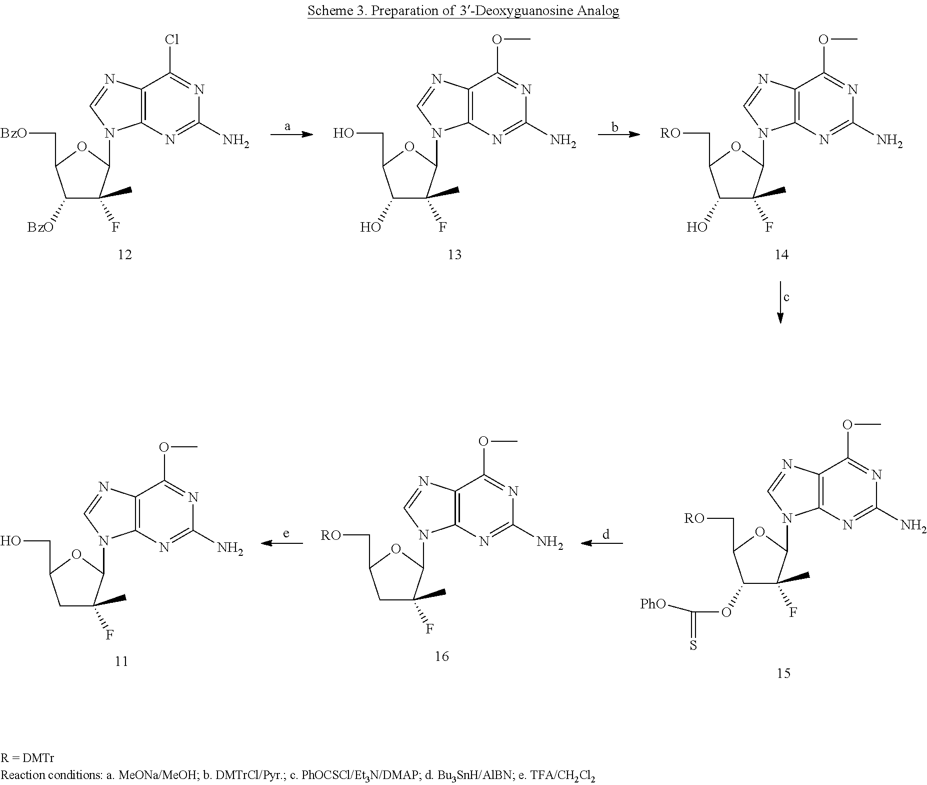

Preparation of phosphoramidate prodrugs of nucleoside 11 was accomplished (Scheme 4) following literature method (WO/2008/121634) as diastereomeric mixture due to the chiral center newly generated at phosphorus. Treatment of POCl₃ with one mole of phenol or alcohol and one mole of Et₃N at −78° C. followed by one mole of amine (or amino acid ester) and one mole of Et₃N gave phosphorus monochloride. Treatment of nucleoside 11 with the newly prepared phosphorus chloride in the presence of N-methylimidazole (NMI) gave the nucleoside phosphoramidate 17 and 18, respectively.

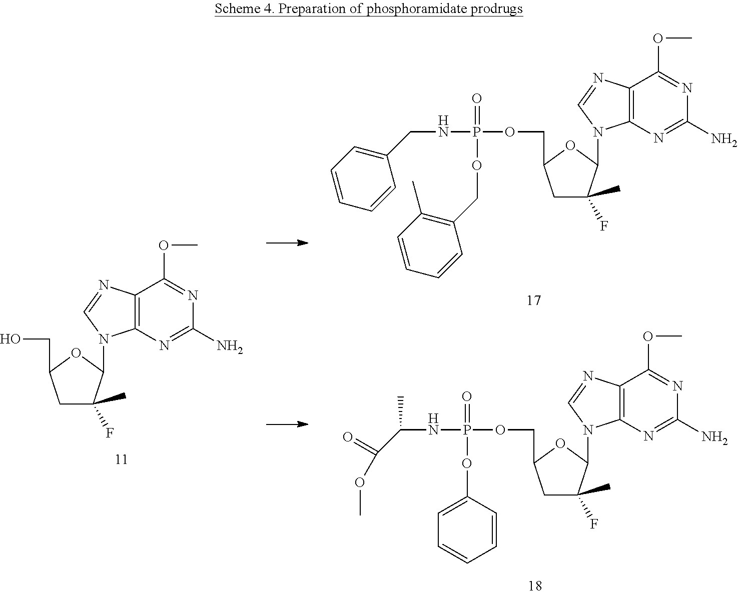 [SMILES: COc1nc(N)nc2c1ncn2C1OC(CO)C[C@@]1(C)F ; COc1nc(N)nc2c1ncn2C1OC(COP(=O)(NCc2ccccc2)OCc2ccccc2C)C[C@@]1(C)F ; COC(=O)[C@H](C)NP(=O)(OCC1C[C@@](C)(F)C(n2cnc3c(OC)nc(N)nc32)O1)Oc1ccccc1]

The preparation of chiral phosphoramidate prodrugs was accomplished by reaction of chiral reagents, such as compound 21 and 23, with nucleoside. As example, treatment of phenyl dichlorophosphate (1 mmol) with amino acid ester hydrochloride 20 and 22 (1 mmol) and triethylamine (2 mmol) at −78° C. followed by reaction of the resulting intermediate with pentafluorophenol (1 mmol) and triethylamine (1 mmol) to give the chiral intermediate isopropyl ester (21) and cyclopentyl ester (23), respectively after recrystallization (Scheme 5). Reaction of nucleoside 11 with chiral reagents 21 and 23 in the presence of t-BuMgCl provided diastereomeric pure phosphoramidate 24 and 25 (Scheme 6), respectively.

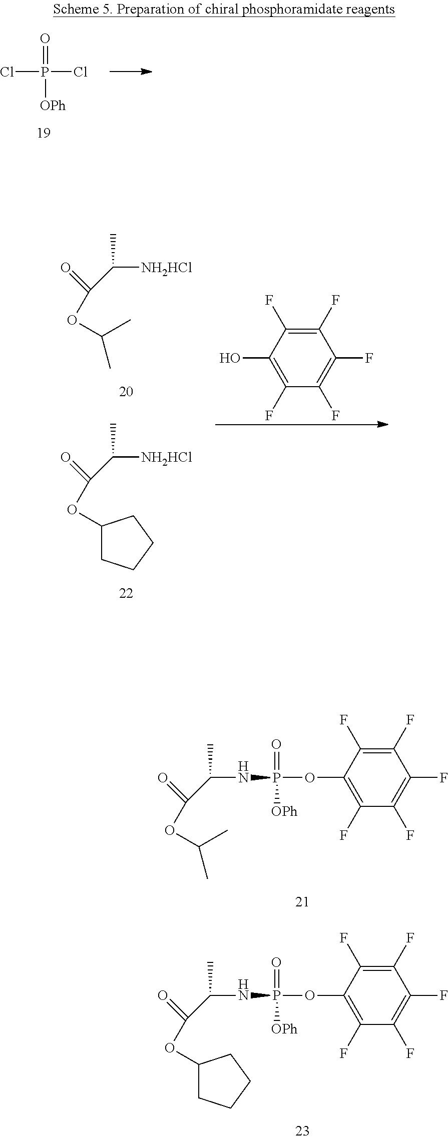

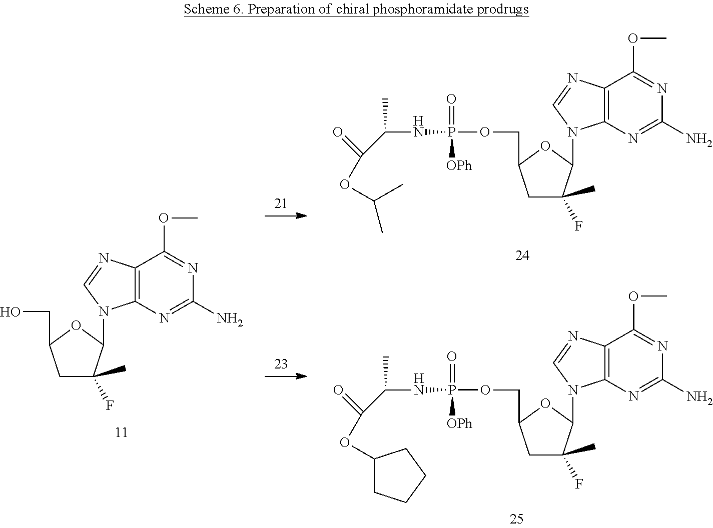 [SMILES: COc1nc(N)nc2c1ncn2C1OC(CO)C[C@@]1(C)F ; COc1nc(N)nc2c1ncn2C1OC(CO[P@@](=O)(N[C@@H](C)C(=O)OC(C)C)Oc2ccccc2)C[C@@]1(C)F ; COc1nc(N)nc2c1ncn2C1OC(CO[P@@](=O)(N[C@@H](C)C(=O)OC2CCCC2)Oc2ccccc2)C[C@@]1(C)F]

Diastereomers of 21 and 23 can be obtained by separation of the mother liquor from recrystallization. These diastereomers of 21 and 23 can be used for the preparation of diastereomers of 24 and 25, respectively.

### Biological Evaluation

The anti-HCV activity and cytotoxicity of compounds disclosed herein were evaluated following patent method (WO/2007/027248).

### Examples

The following examples illustrate the synthesis of representative compounds provided herein. These examples are not intended, nor are they to be construed, as limiting the scope of the claimed subject matter. It will be clear that the scope of claimed subject matter may be practiced otherwise than as particularly described herein. Numerous modifications and variations of the subject matter are possible in view of the teachings herein and, therefore, are within the scope the claimed subject matter.

Product of phosphoramidates prepared herein can be one or a mixture of diasteromers due to the newly formed chiral center of phosphorus and tested as one or a mixture in biological assays.

Single isomers can be obtained by HPLC separation or prepared from chiral intermediates.

Nuclear magnetic resonance (NMR) spectra were recorded on a Bruker advance II 400 MHz and a VarianUnity Plus 400 MHz spectrometers at room temperature, with tetramethylsilane as an internal standard. Chemical shifts (6) are reported in parts per million (ppm), and signals are reported as s (singlet), d (doublet), t (triplet), q (quartet), m(multiplet), or br s (broad singlet).

1. Preparation of Compound 3

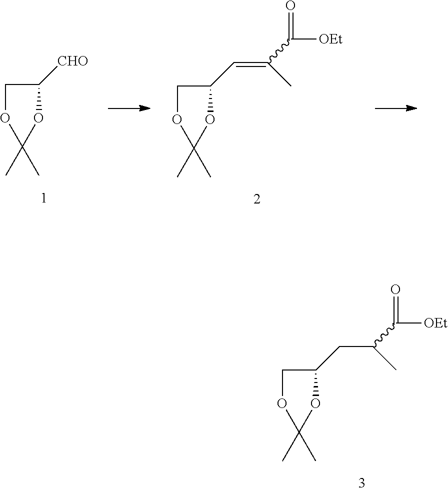

To a solution of (carbethoxyethylidine)triphosphorane (2.0 g, 5.5 mmol) in dry CH₂Cl₂ (10 mL) at room temperature was added dropwise a solution of 2,3-isopropylidene-D-glyceraldehyde (0.94 g, 7.2 mmol) in CH₂Cl₂ (3 mL). The mixture was stirred at room temperature overnight. The reaction mixture was concentrated to dryness, diluted with light petroleum ether (50 mL), and kept at room temperature for 2 h. Triphenylphosphine oxide precipitated was removed by filtration and the filtrate was concentrated to dryness. The residue was purified by silica gel column chromatography (0-5% EtOAc in hexanes) to give compound 2 (0.83 g, 70%) (Carbohydrate Res. 1983, 115, 250). δ_{H} (CDCl₃): δ 1.30 (t, J=6.8 Hz, 3H), 1.41 (s, 3H), 1.45 (s, 3H), 1.89 (d, J=1.2 Hz), 3.63 (t, J=8.0 Hz, 1H), 4.14 (m, 3H), 4.86 (m, 1H), 6.69 (dd, J=1.6, 8.0 Hz) ppm.

To a solution of compound 2 (0.8 g, 3.7 mmol) in MeOH (20 mL) was added Pd/C (100 mg, 10%) and the mixture was stirred under H₂ in balloon for 5 h. The mixture was filtered and the filtrate was concentrated to dryness to give compound 3 (0.80 g, 100%) as a mixture of diastereomers.

2. Preparation of Compound 4 (4′ and 4″)

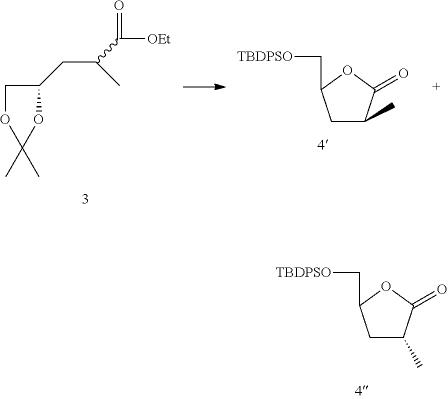

To a solution of compound 3 (20 g, 92 mmol) in EOH (100 mL) and H₂O (20 mL) was added concentrated HCl (37%, 3 mL) and the solution was heated at reflux for 5 h. Solvent was removed and the residue was co-evaporated with pyridine (2×50 mL) then dissolved in pyridine (100 mL). To the solution were added CH₂Cl₂ (200 mL) and then t-butyldiphenylsilylchloride (37.9 g, 138 mmol). The resulting mixture was stirred at room temperature for 20 h. The solvents were evaporated to dryness and the residue was co-evaporated with toluene twice. The residue was dissolved in EtOAc (300 mL) and the mixture was washed with brine and dried over Na₂SO₄. Solvent was removed and the residue was purified by silica gel column (0-30% EtOAc in hexanes) to give a mixture of diastereomers (4, 27.2 g, 80%) which were separated by silica gel column chromatography to give individual isomers. Less polar isomer: δ_{H} (CDCl₃): δ 1.01 (s, 9H), 1.28 (d, J=7.6 Hz, 3H), 2.44 (m, 1H), 2.84 (m, 1H), 3.66 (dd, J=3.6, 11.6 Hz, 1H), 3.85 (dd, J=3.6, 11.6 Hz, 1H), 4.54 (m, 1H), 7.65, 7.42 (mm, 10H). More polar isomer: δ_{H} (CDCl₃): 1.05 (s, 9H), 1.29 (d, J=6.8 Hz, 3H), 1.85 (m, 1H), 2.39 (m, 1H), 2.70 (m, 1H), 2.72 (, dd, J=4.0, 11.2 Hz, 1H), 3.85 (dd, J=3.6, 11.6 Hz, 1H), 4.45 (m, 1H), 7.41-7.66 (m, 10H).

Mixture of 4 (4′ and 4″) can be directly used for the next fluorination without separation.

3. Preparation of Compound 5.

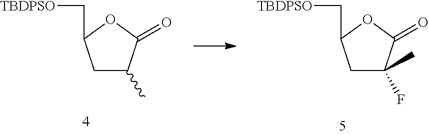

To a solution of compound 4 (3.68 g, 10 mmol) and N-fluorodibenzenesulfonimide (4.73 g, 15 mmol) in THF (50 mL) was added 1M LHMDS in THF (20 mmol, 20 mL) dropwise at −78° C. and the solution was stirred at −78° C. for an additional 2 h and then room temperature for 1 h. The reaction solution was quenched with aq NH₄Cl and the organic solution was washed with brine and dried over Na₂SO₄. Solvent was removed and the residue was purified by silica gel column chromatography (0-20% EtOAc in hexanes) to give compound 5 (2.72 g, 71%). δ_{H} (CDCl₃): δ 1.05 (s, 9H), 1.66 (d, J=22.4 Hz, 3H), 2.44 (m, 2H), 3.70 (dd, J=3.6, 12.0 Hz, 1H), 3.95 (dd, J=3.6, 12.0 Hz, 1H), 4.72 (m, 1H), 7.43-7.65 (mm, 10H). ¹⁹F (CDCl₃): 147 ppm.

4. Preparation of Compound 7

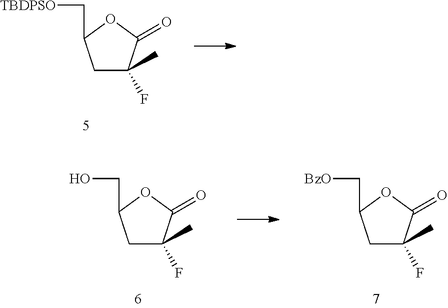

To a solution of compound 5 (3.86 g, 10 mmol) in THF (30 mL) was added TBAF (1.5 eq) and the solution was stirred at rt for 3 h. The solvent was removed and the residue was co-evaporated with pyridine (2×10 mL). The residue was dissolved in pyridine (10 mL) and CH₂Cl₂ (20 mL). To the solution was added BzCl (1.5 eq) and the solution was stirred at room temperature for 3 h. Water (5 mL) was added and the mixture was extracted with CH₂Cl₂ (2×50 mL). Organic solution was dried over Na₂SO₄. Solvent was removed and the residue was co-evaporated with toluene (2×50 mL). The resulting residue was purified by silica gel column chromatography (0-30% EtOAc in hexanes) to give compound 7 as single compound. δ_{H} (CDCl₃): δ 1.68 (d, J=22.4 Hz, 3H), 2.12 (m, 1H), 2.72 (m, 1H), 4.44 (dd, J=5.6, 12.4 Hz. 1H), 4.61 (dd, J=3.2, 12.4 Hz, 1H), 5.00 (m, 1H), 7.48, 7.58, 8.02 (mmm, 5H). 4 (CDCl₃): 149 (m). m/z: 253 [M+H]⁺.

5. Preparation of Lactol 8.

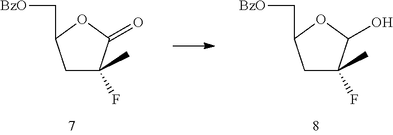

To a solution of compound 7 (2.52 g, 10 mmol) in THF (50 mL) was added a solution of LiAl(t-BuO)₃H in THF (1M, 11 mmol, 11 mL) dropwise and the solution was stirred at −30° C. for 2 h. The reaction was quenched with a solution of NH₄Cl. The mixture was extracted with EtOAc (200 mL) and the organic solution was washed with brine and dried over Na₂SO₄. Solvent was removed and the residue was purified by silica gel column chromatography (0-50% EtOAc in hexanes) to give compound 8 (2.3 g, 92%).

6. Preparation of Purine Nucleoside 10

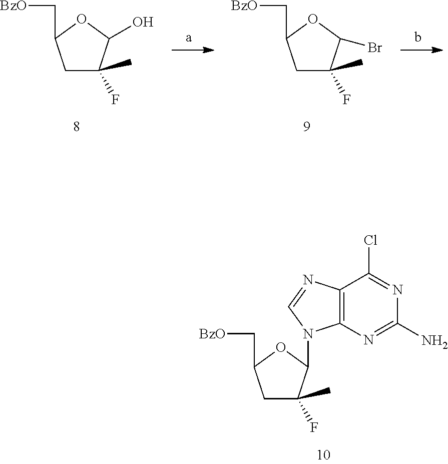

To a solution of triphenylphosphine (3.66 g, 14 mmol) in THF (100 mL) was added lactol 8 (2.54 g, 10 mmol) and the solution was stirred at −20° C. for 15 min. CBr₄ (4.97 g, 15 mmol) was added portion-wise at −20° C. within 30 min. After completion of addition of CBr₄, reaction mixture was stirred at −20° C. for additional 20 min then passed through a silica gel pad. The filtrate was evaporated to dryness to give 1-bromo-α-sugar 9 as major product.

To a suspension of 2-amino-6-chloropurine (4.22 g, 25 mmol) in t-BuOH (100 mL) was added t-BuOK (2.8 g, 25 mmol) portion-wise under N₂. The mixture was stirred at room temperature for an additional 30 min. To the reaction mixture was added α-bromosugar 9 prepared above and dry ACN (80 mL) at room temperature. The mixture was heated to 50° C. over 2 h and stirred at room temperature for 20 h. The reaction was quenched with aq NH₄Cl. The suspended solid was removed by filtration through celite. The filtrate was neutralized by adding 6 N HCl until pH7.0. The mixture was concentrated to dryness and the residue was purified by silica gel column chromatography (0-80% EtOAc in hexances) to give product β-nucleoside 13.

7. Preparation of Compound 11

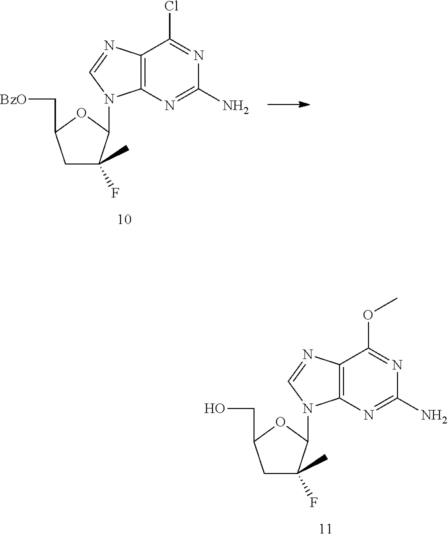

To a solution of compound 10 (4.06 g, 10 mmol) in MeOH (100 mL) was added NaOMe (25% in MeOH, 30 mmol) and the solution was stirred at room temperature for 24 h. Solvent was evaporated and the residue was purified by silica gel column chromatography (0-15% MeOH in CH₂Cl₂) to give compound 11 (2.5 g, 84%). δ_{H} (CD₃OD): 1.20 (d, J=21.6 Hz, 3H), 2.24 (m, 1H), 2.47 (m, 1H), 3.76 (dd, J=3.2, 12.8 Hz, 1H), 4.01 (dd, J=3.2, 12.8 Hz, 1H), 4.48 (m, 1H), 6.15 (d, J=16.8 Hz, 1H), 8.32 (s, 1H). m/z: 298 [M+H]⁺.

8. Preparation of Compound 11 by 3′-Deoxygenation of Nucleoside Precursor (Scheme 3).

To a solution of compound 12 (5.26 g, 10 mmol) in MeOH (100 mL) was added NaOMe (25% in MeOH, 45 mmol) and the solution was stirred at room temperature for 24 h. Solvent was evaporated and the residue was purified by silica gel column chromatography (0-15% MeOH in CH₂Cl₂) to give compound 13 (2.9 g, 93%).

To a solution of 2′-deoxy-2′-fluoro-α-2′-C-methylnucleoside 13 (1.57 g, 5 mmol) in pyridine (20 mL) was added DMTrCl (2.88 g, 7.5 mmol) portion-wise and the solution was stirred at 0° C. for 2 h. Water (10 mL) was added and the mixture was extracted with EtOAc (2×100 mL). The organic solution was washed with brine and dried over Na₂SO₄. Solvent was evaporated to dryness and the residue was co-evaporated with toluene twice. The residue was dissolved in pyridine (20 mL). To the solution were added DMAP (1.8 g, 15 mmol) and triethylamine (1.52 g, 15 mmol), then PhOCSCl (2.59 g, 15 mmol) and the solution was stirred at 0° C. for 1 h and room temperature for 16 h. EtOAc (200 mL) was added and the mixture was washed with brine and dried over Na₂SO₄. Solvent was removed and the residue was dissolved in dry toluene (20 mL). The solution was bubbled with N₂ for 5 min. To the solution was added Bu₃SnH (10 eq) and AIBN (1 mmol), and the solution was heated at 100° C. for 8 h. Solvent was evaporated to dryness under reduced pressure. The residue was dissolved in CH₂Cl₂ (100 mL) and TFA (2 mL) was added. The solution was stirred at room temperature for 3 h. Ammonium hydroxide was added to neutralize the solution to pH7. The mixture was concentrated to dryness and the residue was purified by silica gel column chromatography (0-10% MeOH in CH₂Cl₂) to give 3′-deoxynucleoside 11 (overall yield 50-70%).

9. Preparation of Phosphoramidates 17

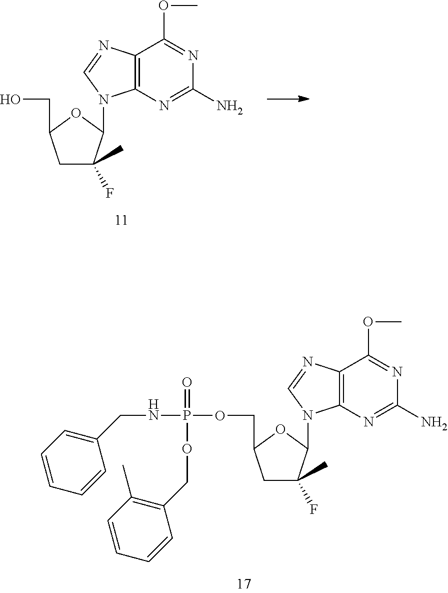 [SMILES: COc1nc(N)nc2c1ncn2C1OC(CO)C[C@@]1(C)F ; COc1nc(N)nc2c1ncn2C1OC(COP(=O)(NCc2ccccc2)OCc2ccccc2C)C[C@@]1(C)F]

To a solution of phosphorus oxychloride (3.07 g, 20 mmol) in THF (40 mL) was added a solution of 2′-methylbenzyl alcohol (2.44 g, 20 mmol) and triethylamine (2.02 g, 20 mmol) in THF (10 mL) at −78° C., and the mixture was stirred at −78° C. for 3 h. To the resulting mixture was added a solution of benzylamine (2.14 g, 20 mmol) and triethylamine (2.02 g, 20 mmol) in THF (10 mL) at −78° C. and the mixture was stirred at −78° C. for 1 h then room temperature for overnight. THF was removed under vacuum and the residue was filtered and washed with ethyl ether (50 mL). The filtrate and washing was evaporated to give crude monochloride which was dissolved in CH₂Cl₂ (10 mL) for the next reaction without further purification. To a suspension of nucleoside 11 (1.49 g, 5 mmol) in CH₂Cl₂ (40 mL) was added N-methylimidazole (NMI, 5 mL) and the mixture was cooled in an ice-bath. To the solution was added a solution of monochloride above and the resulting solution was stirred in an ice-bath for 3 h. Water (5 mL) was added and the mixture was extracted with EtOAc (2×200 mL). The organic solution was washed with 0.5 N HCl solution, aq NaHCO₃, brine, and dried over Na₂SO₄. The solvent was removed under vacuum and the residue was purified by silica gel chromatography (0-8% MeOH in CH₂Cl₂) to give compound 17 (1.25 g, 43.9%). δ_{H} (CDCl₃): 1.18, 1.22 (dd, J=21.6 Hz, 3H), 2.2-2.8 (m, 5H), 3.18 (m, 1H), 4.0-4.2 (m, 6H), 4.60 (m, 2H), 5.10 (m, 2H), 5.16, 5.28 (ss, 1H), 6.00 (dd, J=17.6 Hz, 1H), 7.10 (m, 9H), 7.70 (s, 1H). m/z: 571 [M+H]⁺.

10. Preparation of Phosphoramidates 18

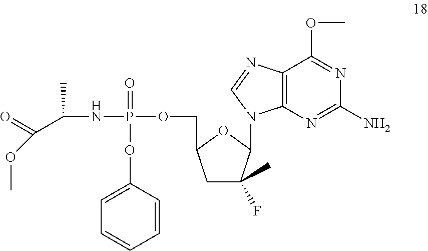 [SMILES: COC(=O)[C@H](C)NP(=O)(OCC1C[C@@](C)(F)C(n2cnc3c(OC)nc(N)nc32)O1)Oc1ccccc1]

From L-alanyl methyl ester, applying similar reaction used for 17 provided compound 18. δ_{H} (CD₃OD): 1.20, 1.40 (mm, 6H), 2.20-2.80 (m, 2H), 3.59 (m, 1H), 3.68, 3.69 (ss, 3H), 4.06, 4.26 (ss, 3H), 4.29 (m, 1H), 4.62 (m, 1H), 4.74 (m, 1H), 5.11, 5.30 (ss, 2H), 6.00 (dd, J=17.6 Hz, 1H), 7.25 (m, 5H), 7.66, 7.73 (ss, 1H). m/z: 539 [M+H]^{±}.

11. Preparation of Chiral Phosphorus Reagents 21

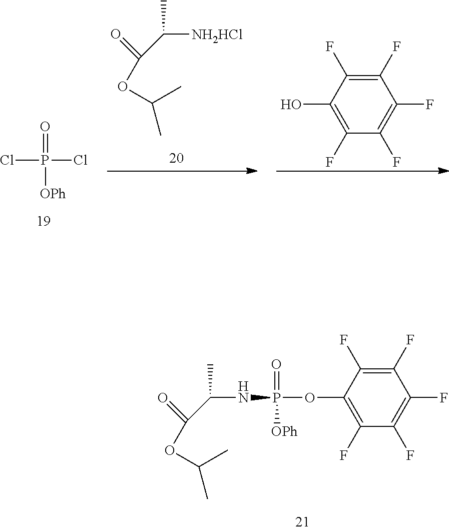

To a solution of PhOPOCl₂ (19, 6.14 g, 40 mmol) in CH₂Cl₂ (80 mL) was added L-alanyl isopropyl ester hydrochloride (20, 6.7 g, 40 mmol) then a solution of Et₃N (80 mmol) in CH₂Cl₂ (10 mL) at −78° C. The mixture was stirred at room temperature for overnight. To the mixture was added a solution of pentafluorophenol (7.36 g, 40 mmol) and Et₃N (80 mmol) in CH₂Cl₂ (10 mL) and the mixture was stirred at room temperature for 4 h. Filtered and the cake was washed with CH₂Cl₂. The filtrate was evaporated and the residue was dissolved in EtOAc (200 mL). The solution was washed with Aq. NaHCO₃, brine and dried over Na₂SO₄. Solvent was evaporated and the residue was purified by silica gel column chromatography (5-50% EtOAc in hexanes) to give a mixture of diastereomers of compound 21. The mixture was recrystallized from EtOAc-hexane to give single isomer 21 (25-40% yield). δ_{H} (CDCl₃): m/z: 539 [M+H]⁺.

12. Preparation of Chiral Phosphorus Reagents 23

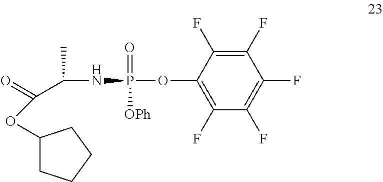 [SMILES: C[C@H](N[P@](=O)(Oc1ccccc1)Oc1c(F)c(F)c(F)c(F)c1F)C(=O)OC1CCCC1]

From L-alanyl cyclopentyl ester hydrochloride 22, applying similar reaction used for 21 produced compound 23. δ_{H} (CDCl₃): m/z: 539 [M+H]⁺.

13. Preparation of Chiral Phosphoramidate Prodrugs 24

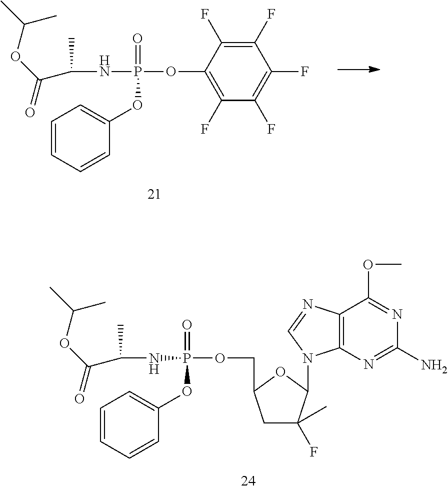 [SMILES: CC(C)OC(=O)[C@H](C)N[P@](=O)(Oc1ccccc1)Oc1c(F)c(F)c(F)c(F)c1F ; COc1nc(N)nc2c1ncn2C1OC(CO[P@@](=O)(N[C@@H](C)C(=O)OC(C)C)Oc2ccccc2)CC1(C)F]

To a mixture of reagent 21 (1.36 g, 3.0 mmol) and purine nucleoside 11 (0.594 g, 2.0 mmol) in THF (50 mL) was added a solution of t-BuMgCl (1 M in THF, 6 mmol) and the mixture was stirred at room temperature for 2 h. EtOAc (200 mL) was added and the mixture was washed with brine and dried over Na₂SO₄. Solvent was removed and the residue was purified by silica gel column chromatography (5% MeOH in CH₂Cl₂) to give compound 24 as white foam (60-80% yield). m/z: 539 [M+H]⁺.

14. Preparation of Chiral Phosphoramidate Prodrugs 25

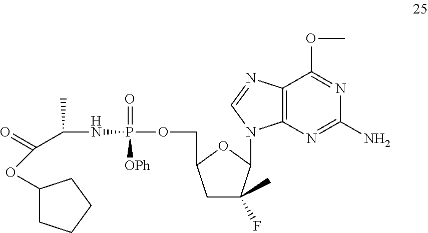 [SMILES: COc1nc(N)nc2c1ncn2C1OC(CO[P@@](=O)(N[C@@H](C)C(=O)OC2CCCC2)Oc2ccccc2)C[C@@]1(C)F]

From chiral reagent 23, applying similar reaction used for 24 produced compound 25. δ_{H} (400 MHz, CDCl₃): 7.66 (s, 1H), 7.15-7.35 (m, 5H), 5.96 (d, J=17.6 Hz, 1H), 5.33 (br s, 2H), 5.14 (m, 1H), 4.80 (m, 1H), 4.61 (m, 1H), 4.27 (m, 1H), 4.06 (s, 3H), 3.97 (m, 1H), 3.62 (t, J=10.4 Hz, 1H), 2.80 (m, 1H), 2.20 (m, 1H), 1.80 (m, 2H), 1.60 (m, 8H), 1.34 (d, J=6.8 Hz, 3H), 1.20 (d, J=22.0 Hz, 3H). m/z: 539 [M+H]⁺. ³¹P (CDCl₃): 4.288 ppm.

15. HCV Replicon Assay

The anti-HCV activity and toxicity of the exemplary compounds can be tested in two biological assays—a cell-based HCV replicon assay and cytotoxicity assay (WO 2007/027248).

I. Anti-HCV Assay

A human hepatoma cell line (Huh-7) containing replicating HCV subgenomic genotype 1b replicon with a luciferase reporter gene (luc-ubi-neo) was used to evaluate anti-HCV activity of the compounds. In this assay, the level of luciferase signal correlates with the viral RNA replication directly. The HCV replicon-reporter cell line (NK/luc-ubi-neo) was cultured in DMEM medium supplemented with 10% fetal bovine serum and 0.5 mg/ml Geneticin (G418). Cells were maintained in a subconfluent state to ensure high levels of HCV replicon RNA synthesis.

To evaluate the antiviral activity of compounds, serial dilutions were prepared with concentrations ranging from 0.14 to 300 μM. Diluted compounds were transferred to a 96-well plate followed by the addition of replicon cells (6000 cells per well). Cells were incubated with the compounds for 48 h after which luciferase activity was measured. Reduction of luciferase signal reflected the decrease of HCV replicon RNA in the treated cells and used to determine the EC₅₀ value (concentration which yielded a 50% reduction in luciferase activity).

II. Cytotoxicity Assay A Huh-7 cell line carrying a luciferase reporter gene (driven by a HIV LTR promoter) stably integrated into the chromosome was used to analyze the cytotoxic effect of the selected compounds. This cell line (LTR-luc) was maintained in DMEM medium with 10% FBS. Design of the cytotoxicity assay was similar to that of the HCV replicon assay. Reduction of luciferase activity in the treated cells correlated with the cytotoxic effect of the test compound and was used to calculate the CC₅₀ value (concentration that inhibited cell growth by 50%).

The biological results of the selected compounds are summarized in Table 1.

TABLE 1
Activity of exemplary compound in replicon assay (genotype 1b)

| Compd. | EC₅₀ (μM) | CC₅₀ (μM) |
|---|---|---|
| 11 | >100 | >100 |
| 17 | <1 | 52.2 |
| 18 | <1 | >100 |
| 25 | 0.58 | >100 |

EC₅₀ is concentration of drug inhibiting HCV by 50%.
CC₅₀ is concentration of drug inhibiting cellular growth by 50%.

The results of anti-HCV activity of the selected nucleoside prodrugs summarized in Table 1 indicated that these nucleoside phosphate prodrugs 17, 18 and 25 demonstrated potent anti-HCV activity without significant cytotoxicity, which warrants further investigation towards the development of novel nucleosides or their prodrugs disclosed herein as anti-HCV agents.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the following claims. All references cited hereby are incorporated by reference in their entirety.

1. A compound of formula (I): 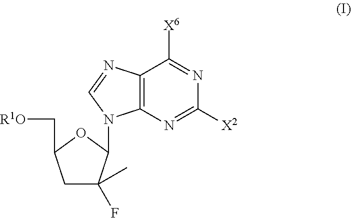
or a pharmaceutically acceptable prodrug, salt, or solvate thereof, wherein:
R¹ is selected from H, monophosphate, diphosphate, triphosphate, acyl (R²CO—), R²OC(O)—, R²NHC(O)—, and R^{a}R^{b}NC(O)—;
R² is selected from alkyl, alkenyl, alkynyl, aryl, benzyl, cycloalkyl, heterocyclyl, and heteroaryl;
R^{a} and R^{b} are independently selected from alkyl, alkenyl, alkynyl, aryl, benzyl, cycloalkyl, heterocyclyl, and heteroaryl, or alternatively, R^{a} and R^{b} together with the nitrogen atom (N) to which they are attached form a 4- to 7-membered ring;
X² is selected from H, NH₂, and halogen (I, Br, Cl, F); and
X⁶ is selected from H, —OH, —OMe, —OEt, —SMe, alkyloxy, aryloxy, cycloalkyloxy, alkylthio, arylthio, cycloalkylthio, alkylamino, dialkylamino, arylamino, diarylamino, arylalkylamino, cycloalkylamino, and cyclopropylamino, wherein dialkyl portion of the dialkylamino group optionally forms a ring along with the nitrogen atom of the amino group wherein any of the amino and hydroxyl groups are optionally protected.

2. The compound of claim 1, or its pharmaceutically acceptable prodrug or salt thereof, selected from compounds of formulae: 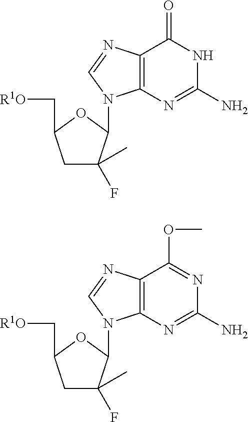
wherein R¹ is defined as in claim 1.

3. The compound of claim 1, or its pharmaceutically acceptable prodrug or salt thereof, wherein R¹ is H, monophosphate, diphosphate, or triphosphate.

4. The compound of claim 1, or a pharmaceutically acceptable salt thereof, selected from stable phosphate prodrug compounds of formulae: 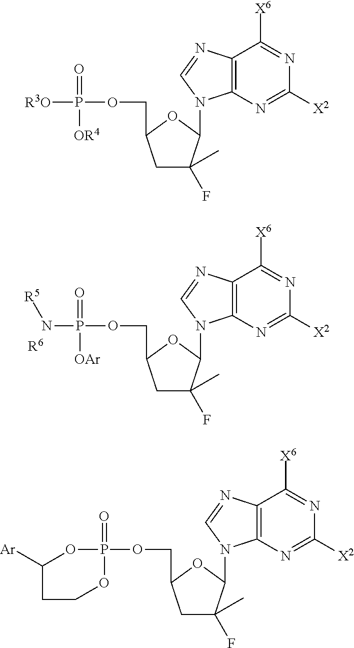
wherein:
X² and X⁶ are defined as in claim 1;
R³ and R⁴ are independently selected from alkyl, cycloalkyl, aryl, benzyl, and substituents characterized by formulae: 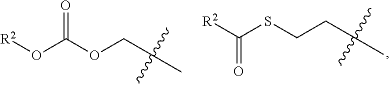
wherein: R² is defined as in claim 1;
Ar is unsubstituted or substituted aryl or heteroaryl;
R⁵ and R⁶ are independently selected from alkyl, alkenyl, alkynyl, aryl, benzyl, cycloalkyl, heterocyclyl, heteroaryl, or alternatively, R⁵ and R⁶ together form a 4- to 7-membered ring along with the nitrogen atom (N) to which they are attached;
or alternatively, R⁵R⁶N is selected from amino acid residues and aminoalcohol groups of formulae: 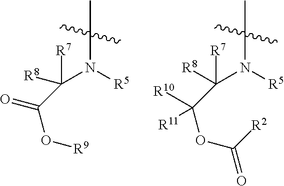
wherein:
R² is defined as in claim 1; and
R⁷, R⁸, R⁹, R¹⁰ and R¹¹ are each independently selected from alkyl, alkenyl, alkynyl, aryl, cycloalkyl, heterocyclyl, and heteroaryl, each substituted or unsubstituted, wherein R⁷ and R⁸ together or R¹⁰ and R¹¹ together, along with the carbon atoms to which they are attached, can optionally independently form a 3- to 7-membered ring.

5. The compound of claim 1, or a pharmaceutically acceptable prodrug or salt thereof, selected from diastereomers of a stable phosphate prodrug according to formula: 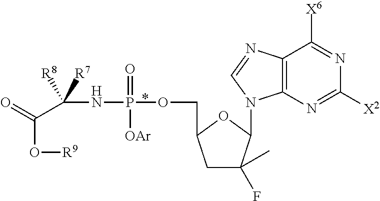
and mixtures thereof, wherein X² and X⁶ are defined as in claim 1;
Ar is unsubstituted or substituted aryl or heteroaryl; and
R⁷, R⁸, and R⁹ are each independently selected from alkyl, alkenyl, alkynyl, aryl, cycloalkyl, heterocyclyl, and heteroaryl, each substituted or unsubstituted, or alternatively R⁷ and R⁸, along with the carbon atoms to which they are attached, form a 3- to 7-membered ring.

6. The compound of claim 1, or its pharmaceutically acceptable prodrug or salt thereof, selected from stable phosphate prodrugs and diastereomers thereof according to formulae: 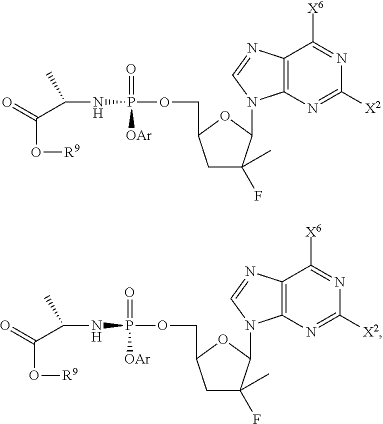
wherein X² and X⁶ are defined as in claim 1;
Ar is unsubstituted or substituted aryl or heteroaryl; and
R⁹ is selected from alkyl, alkenyl, alkynyl, aryl, cycloalkyl, heterocyclyl, and heteroaryl, each substituted or unsubstituted.

7. The compound of claim 1, or a pharmaceutically acceptable prodrug or salt thereof, selected from stable phosphate prodrugs and diastereomers thereof according to formulae: 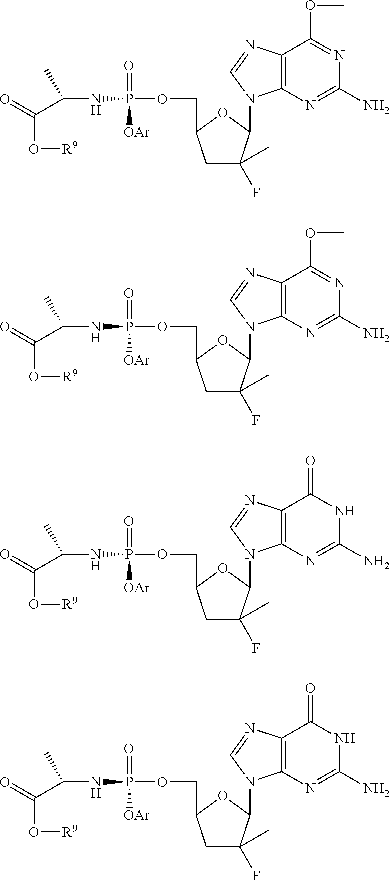
wherein Ar is unsubstituted or substituted aryl or heteroaryl; and
R⁹ is selected from alkyl, alkenyl, alkenyl, aryl, cycloalkyl, heterocyclyl, and heteroaryl, each substituted or unsubstituted.

8. The compound of claim 1, or a pharmaceutically acceptable prodrug or salt thereof, selected from compounds of formulae:

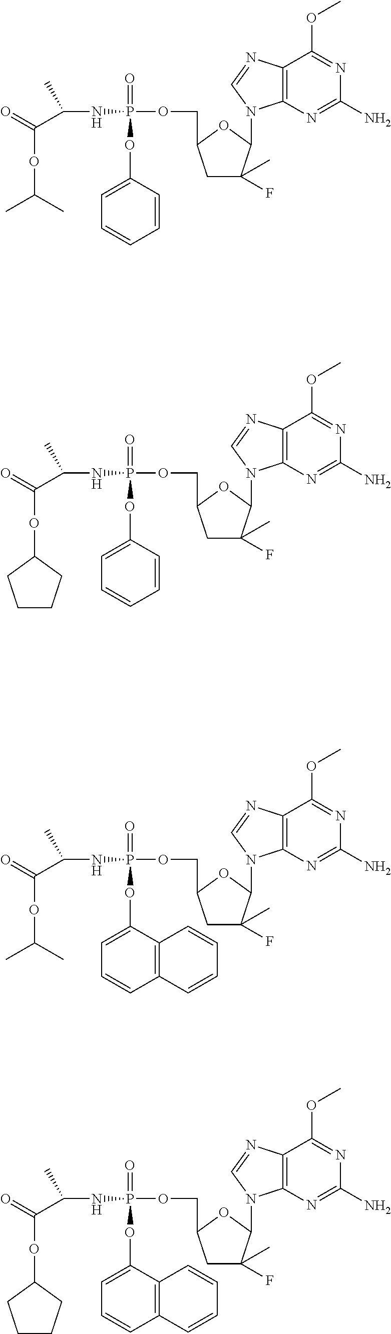 [SMILES: COc1nc(N)nc2c1ncn2C1OC(CO[P@@](=O)(N[C@@H](C)C(=O)OC(C)C)Oc2ccccc2)CC1(C)F ; COc1nc(N)nc2c1ncn2C1OC(CO[P@@](=O)(N[C@@H](C)C(=O)OC2CCCC2)Oc2ccccc2)CC1(C)F ; COc1nc(N)nc2c1ncn2C1OC(CO[P@@](=O)(N[C@@H](C)C(=O)OC(C)C)Oc2cccc3ccccc23)CC1(C)F ; COc1nc(N)nc2c1ncn2C1OC(CO[P@@](=O)(N[C@@H](C)C(=O)OC2CCCC2)Oc2cccc3ccccc23)CC1(C)F]

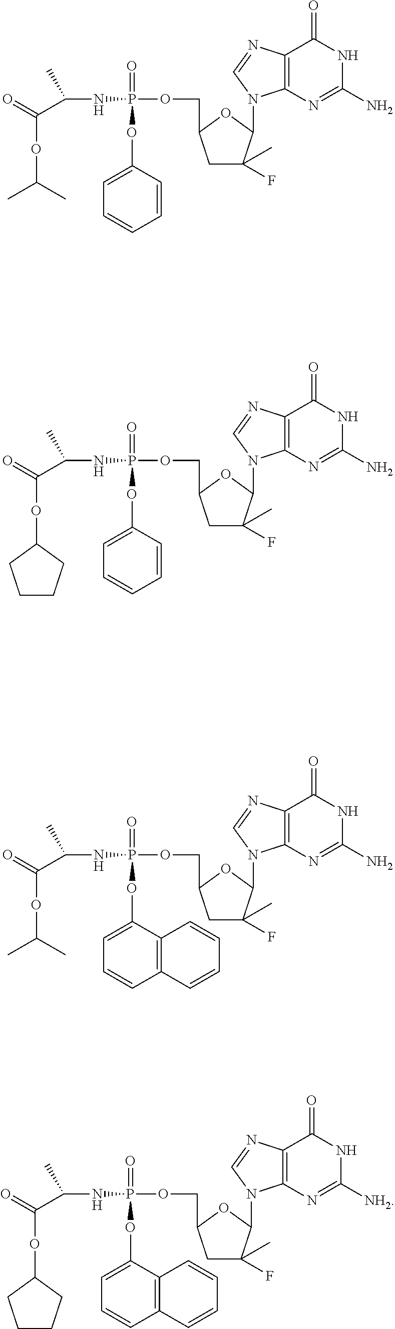 [SMILES: CC(C)OC(=O)[C@H](C)N[P@](=O)(OCC1CC(C)(F)C(n2cnc3c(=O)[nH]c(N)nc32)O1)Oc1ccccc1 ; C[C@H](N[P@](=O)(OCC1CC(C)(F)C(n2cnc3c(=O)[nH]c(N)nc32)O1)Oc1ccccc1)C(=O)OC1CCCC1 ; CC(C)OC(=O)[C@H](C)N[P@](=O)(OCC1CC(C)(F)C(n2cnc3c(=O)[nH]c(N)nc32)O1)Oc1cccc2ccccc12 ; C[C@H](N[P@](=O)(OCC1CC(C)(F)C(n2cnc3c(=O)[nH]c(N)nc32)O1)Oc1cccc2ccccc12)C(=O)OC1CCCC1]

9. A pharmaceutical composition comprising a compound of claim 1, or a pharmaceutically acceptable prodrug or salt thereof, and a pharmaceutically acceptable carrier.

10. The pharmaceutical composition of claim 9, further comprising a second or more anti-HCV agents.

11. A method of treating hepatitis C virus infection in a patient, comprising administering a therapeutically effective amount of a compound of claim 1 to the patient.

12. The method of claim 11, in combination with administration of a second or more anti-HCV agents to the patient.

13. A method of treating hepatitis C virus infection, comprising administering a therapeutically effective amount of a pharmaceutical composition of claim 9, to a patient in need of the treatment.

14. The method of claim 13, further comprising administering to the patient a therapeutically effective amount of a second or more anti-HCV agents.

15. A method of making a 2-fluoro-2-C-methyl-nucleoside compound of formula 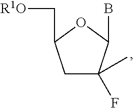
wherein R¹ is as defined in claim 1 and B is a purine base, comprising the steps of:
a. preparing a 5-protected 2-fluoro-α-2-C-methyl-lactone compound of formula: 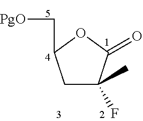
through stereospecific fluorination of a lactone compound of formula: 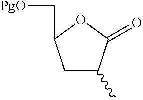
using (PhSO₂)NF or other fluorinating reagents in the presence of base;
b. reducing the 5-protected 2-fluoro-α-2-C-methyl-lactone with a reducing reagent to provide 5-protected 2-fluoro-α-2-C-methyl-1-α-lactol compound of formula: 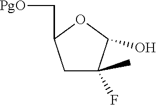
c. converting the 5-protected 2-fluoro-α-2-C-methyl-1-α-lactol compound stereoselectively to an α-intermediate comprising a leaving group L (such as Br) of formula: 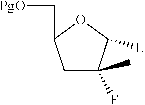
d. reacting the 1-L-α-intermediate with purine or modified purine in the presence of base to stereoselectively produce a β-nucleoside compound of formula: 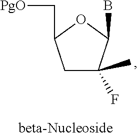
wherein Pg is H or a protecting group.

16. An intermediate useful for the preparation of a compound of claim 1, selected from compounds of formulae: 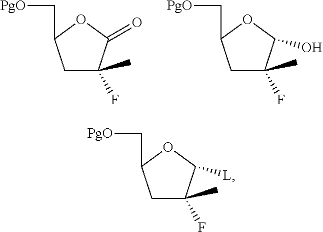
wherein Pg is H or a protecting group, and L is a leaving group.

17. The compound of claim 2, or its pharmaceutically acceptable prodrug or salt thereof, wherein R¹ is H, monophosphate, diphosphate, or triphosphate.

18. The compound of claim 4, or a pharmaceutically acceptable prodrug or salt thereof, selected from diastereomers of a stable phosphate prodrug according to formula: 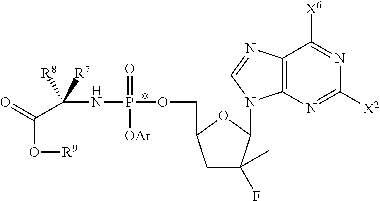
and mixtures thereof, wherein Ar, R⁷, R⁸, R⁹, X² and X⁶ are defined as in claim 4.

19. The method of claim 15, wherein said 2-fluoro-2-C-methyl-nucleoside compound is a compound of claim 1.
